SEQUENCE LISTING (4 sequences)

SEQ ID NO: 1 - DNA, 1120 bp, Corynebacterium glutamicum
  CDS (244)..(852)
acacccatcatttgtcatgtccaactctttcgccgatcagaccattgcgcagatcgaactgttccaaaacgaaggacagtacgagaacgaggtctaccgtctgcctaaggttctcgacgaaaaggtggcacgcatccacgttgaggctctcggcggtcagctcaccgaactgaccaaggagcaggctgagtacatcggcgttgacgttgcaggcccattcaagccggagcactaccgctactaatgattgtcagcattgagggaatcgacggcgccggcaaaaacaccctggtttcggcattaacgcaggttattgatgcaaaagtccttgcattcccacgttatgaaacctcgattcacgcccaattggccgcggaagcactccacggccgcatgggcgacctcaccgacagcgcctacgccatggccacgcttttcgccctcgaccgccacttcgcgattgatgacttaaatgcgcccggcgtggtgctgctcgaccgatacgtcgcctccaacgcggcttataccgccgccagattgcttgacgacgacgccccccgctgggttgccgacctggaattcgggcggcttgggctcccacgtccgacgcttcaagtgttgttggatacccccgcggaggtagcgcaagatagggctagacgtcgagaagcgcttgactccgcgcgtgcgcgggaccgctatgaatcggattcggcgctgcagcaacgcaccgccgagcactatcgccgcctcgcggcggacaactgggaatcaccgtggatcgtggttgcccctgatgaagaccccggccacgttgcgcagagaatcgtggaattcctgggtactataaactaatcccaattagcaggaaggattctcatgtcacagaaaattctcgtggttgatgatgatcccgccatctccgagatgctcaccatcgtgctcagcgcagaaggctttgacaccgtagctgtcaccgacggcgcactcgccgtggaaaccgcctcccgggaacaaccggatctgattttgctcgacttgatgcttccaggcatgaacggcatcgacatttgtcgcctcatccgccaagaatcctccgtacccatcatcatgctcaccg

SEQ ID NO: 2 - protein, 203 aa, Corynebacterium glutamicum
MIVSIEGIDGAGKNTLVSALTQVIDAKVLAFPRYETSIHAQLAAEALHGRMGDLTDSAYAMATLFALDRHFAIDDLNAPGVVLLDRYVASNAAYTAARLLDDDAPRWVADLEFGRLGLPRPTLQVLLDTPAEVAQDRARRREALDSARARDRYESDSALQQRTAEHYRRLAADNWESPWIVVAPDEDPGHVAQRIVEFLGTIN

SEQ ID NO: 3 - DNA, 28 bp, Artificial Sequence
  DNA Primer for PCR
tgggtaccattcaagccggagcactacc

SEQ ID NO: 4 - DNA, 28 bp, Artificial Sequence
  DNA Primer for PCR
gatctagacagcgccgaatccgattcat

(19) United States
(12) Patent Application Publication
(10) Pub. No.: US 2002/0137065 A1
(43) Pub. Date: Sep. 26, 2002

(54) Nucleotide sequences which code for the tmk gene

(75) Inventors: Mike Farwick, Bielefeld (DE); Klaus Huthmacher, Gelnhausen (DE); Achim Marx, Bielefeld (DE); Walter Pfefferle, Halle (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 09/955,203

(22) Filed: Sep. 19, 2001

(30) Foreign Application Priority Data
Sep. 19, 2000 (DE) 100 46 235.9
Aug. 16, 2001 (DE) 101 40 095.0

(51) Int. Cl. C12Q 1/68; C07H 21/04; C12N 9/12; C12P 13/04; C12N 1/21; C12P 21/02

(52) U.S. Cl. 435/6; 435/106; 435/194; 435/69.1; 435/252.3; 435/320.1; 536/23.2

(57) ABSTRACT

The invention relates to polynucleotides comprising polynucleotide sequences corresponding to the tmk gene and parts thereof that encode polypeptide sequences and parts thereof possessing varying degrees of thymidylate kinase activity, methods for preparation of L-amino acids, and methods of screening and amplifying polynucleotides encoding polypeptide sequences which comprise varying degrees of thymidylate kinase activity.

### CROSS-REFERENCE TO RELATED APPLICATIONS

The present application claims priority to German Application No. DE 10046235.9, filed Sep. 19, 2000, and German Application No. DE 10140095.0, filed Aug. 16, 2001. The entire contents of both applications are incorporated herein by reference.

### BACKGROUND OF INVENTION

### FIELD OF THE INVENTION

The invention relates to polynucleotides comprising polynucleotide sequences corresponding to the tmk gene and parts thereof that encode polypeptide sequences and parts thereof possessing varying degrees of thymidylate kinase activity, methods for preparation of L-amino acids, and methods of screening and amplifying polynucleotides encoding polypeptide sequences which comprise varying degrees of thymidylate kinase activity.

### DISCUSSION OF THE BACKGROUND

L-Amino acids, in particular L-lysine, are used in human medicine and in the pharmaceuticals industry, in the foodstuffs industry, and, very particularly, in animal nutrition.

It is known that amino acids are prepared by fermentation from strains of Coryneform bacteria, in particular Corynebacterium glutamicum. Because of their great importance, work is constantly being undertaken to improve the preparation processes. Improvements to the process can relate to fermentation measures, such as, stirring and supply of oxygen, or the composition of the nutrient media, such as, the sugar concentration during the fermentation, or the working up to the product form by, for example, ion exchange chromatography, or the intrinsic output properties of the microorganism itself.

Methods of mutagenesis, selection, and mutant selection are used to improve the output properties of these microorganisms. Strains which are resistant to antimetabolites or are auxotrophic for metabolites of regulatory importance and which produce amino acids are obtained in this manner.

Methods of the recombinant DNA technique have also been employed for some years for creating Coryneform bacterium strains, which produce L-amino acid by amplifying individual amino acid biosynthesis genes and investigating the effect on the amino acid production. During the time preceding the present invention, however, it was not known that attenuated expression of the tmk gene would improve L-amino acid production yields.

### SUMMARY OF THE INVENTION

An object of the present invention is to provide novel measures for improved preparation of L-amino acids or amino acids where these amino acids include L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine, including their salts (such as monohydrochloride or lysine sulfate).

One object of the present invention is a novel process for improving fermentative preparation of the L-amino acids, L-Lysine in particular. This process includes enhanced bacteria, preferably from Coryneform bacteria, which express attenuated amounts of thymidylate kinase, which is encoded by the tmk gene.

Another object of the present invention is to provide such a bacterium, preferably from Coryneform bacteria, which expresses attenuated tmk gene products.

Another e object of the present invention is to provide such a bacterium, preferably from Coryneform bacteria, which expresses attenuated thymidylate kinase activity.

Another object of the present invention is to provide a polynucleotide sequence encoding a polypeptide sequence with thymidylate kinase activity. One embodiment of such a sequence is the polynucleotide sequence of SEQ ID NO. 1.

Another object of the present invention is a method of making thymidylate kinase or a polypeptide having thymidylate kinase activity. One embodiment of such a sequence is the polypeptide sequence of SEQ ID NO. 2.

Other objects of the present invention include methods of detecting polynucleotides that are homologous to SEQ ID NO: 1 or those polynucleotides encoding polypeptides that have having thymidylate kinase activity, methods of making such polynucleotides encoding such polypeptides, and methods of making such polypeptides.

The above descriptions highlight certain aspects and embodiments of the present invention. Additional objects, aspects, and embodiments of the present invention follow in the detailed description of the present invention considered together with the Figures.

### BRIEF DESCRIPTION OF THE FIGURES

FIG. 1: Map of the plasmid pXK99E,

FIG. 2: Map of the plasmid pXK99Etmk.

### DETAILED DESCRIPTION OF THE INVENTION

Unless specifically defined, all technical and scientific terms used herein have the same meaning as commonly understood by a skilled artisan of molecular biology.

“Isolated” refers to a material, i.e. a polynucleotide separated out of its natural environment.

“Polynucleotide” in general relates to polyribonucleotides and polydeoxyribonucleotides, it being possible for these to be non-modified RNA or DNA or modified RNA or DNA.

The term “attenuation” in this connection describes the reduction or elimination of the intracellular activity of one or more enzymes (proteins) in a microorganism which are coded by the corresponding DNA, for example, by using a weak promoter or using a gene or allele which codes for a corresponding enzyme with a low activity or inactivates the corresponding gene or enzyme (protein), and optionally combining these measures. By attenuation measures, the activity or concentration of the corresponding protein is in general reduced to 0 to 75%, 0 to 50%, 0 to 25%, 0 to 10% or 0 to 5% of the activity or concentration of the wild-type protein or of the activity or concentration of the protein in the starting microorganism.

“Polypeptides” are understood as meaning peptides or proteins, which comprise two or more amino acids, bonded via peptide bonds.

The term “enhancement” in this connection describes an increase in the intracellular activity of one or more enzymes (proteins) in a microorganism which are coded by the corresponding DNA, for example, by increasing the number of copies of the gene or genes, using a potent promoter or using a gene or allele which codes for a corresponding enzyme (protein) having a high activity, and optionally combining these measures.

Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. Further, the materials, methods, and examples are illustrative only and are not intended to be limiting.

Reference is made to standard textbooks of molecular biology that contain definitions and methods and means for carrying out basic scientific techniques, encompassed by the present invention. See, for example, Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989) and various references cited therein.

The invention provides an isolated polynucleotide from Coryneform bacteria, comprising a polynucleotide sequence, which codes for the tmk gene, chosen from the group consisting of

(a) polynucleotide which is identical to the extent of at least 70% to a polynucleotide which codes for a polypeptide, which comprises the amino acid sequence of SEQ ID NO. 2,

(b) polynucleotide which codes for a polypeptide, which comprises an amino acid sequence, which is identical to the extent of at least 70% to the amino acid sequence of SEQ ID NO. 2,

(c) polynucleotide which is complementary to the polynucleotides of a) or b), and

(d) polynucleotide comprising at least 15 successive nucleotides of the polynucleotide sequence of a), b) or c), the polypeptide preferably has the activity of thymidylate kinase.

The invention also provides the above-mentioned polynucleotide, this preferably being a DNA which is capable of replication, comprising:

(i) the nucleotide sequence, shown in SEQ ID NO. 1, or

(ii) at least one sequence which corresponds to sequence (i) within the range of the degeneration of the genetic code, or

(iii) at least one sequence which hybridizes with the sequences complementary to sequences (i) or (ii), and optionally

(iv) sense mutations of neutral function in (i).

The invention also provides:

(a) a polynucleotide, in particular DNA, which is capable of replication and comprises the nucleotide sequence as shown in SEQ ID NO. 1;

(b) a polynucleotide, which codes for a polypeptide which, comprises the amino acid sequence as shown in SEQ ID NO. 2;

(c) a vector containing parts of the polynucleotide according to the invention, but at least 15 successive nucleotides of the sequence claimed,

(d) Coryneform bacteria in which the tmk gene is attenuated, in particular by an insertion or deletion.

The invention also provides polynucleotides with a polynucleotide sequence which comprises the complete tmk gene or parts thereof, obtainable by screening by means of hybridization of a corresponding gene library of a Coryneform bacterium with a probe which comprises the sequence of the polynucleotide according to SEQ ID NO.1 or a fragment thereof, and isolation of the polynucleotide sequence mentioned.

The present invention provides polynucleotides which comprise the sequences according to the invention are suitable as hybridization probes for RNA, cDNA and DNA, in order to isolate, in the full length, nucleic acids or polynucleotides or genes which code for thymidylate kinase or to isolate those nucleic acids or polynucleotides or genes which have a high similarity with the sequence of the tmk gene. They are also suitable for incorporation into so-called “arrays”, “micro arrays” or “DNA chips” in order to detect and to determine the corresponding polynucleotides.

Polynucleotides, which comprise the sequences according to the invention, are furthermore suitable as primers, which code for thymidylate kinase can be prepared by the polymerase chain reaction (PCR).

Such oligonucleotides which serve as probes or primers comprise at least 25, 26, 27, 28, 29 or 30, preferably at least 20, 21, 22, 23 or 24, very particularly preferably at least 15, 16, 17, 18 or 19 successive nucleotides. Oligonucleotides which have a length of at least 31, 32, 33, 34, 35, 36, 37, 38, 39 or 40 or at least 41, 42, 43, 44, 45, 46, 47, 48, 49 or 50 nucleotides are also suitable. Oligonucleotides with a length of at least 100, 150, 200, 250 or 300 nucleotides are optionally also suitable.

The polynucleotides according to the invention include a polynucleotide according to SEQ ID NO. 1 or a fragment prepared therefrom and also those which are at least 70% to 80%, preferably at least 81% to 85%, particularly preferably at least 86% to 90%, and very particularly preferably at least 91%, 93%, 95%, 97% or 99% identical to the polynucleotide according to SEQ ID NO. 1 or a fragment prepared therefrom.

The polypeptides according to the invention include a polypeptide according to SEQ ID NO. 2, in particular those with the biological activity of thymidylate kinase, and also those which are at least 70% to 80%, preferably at least 81% to 85%, particularly preferably at least 86% to 90%, and very particularly preferably at least 91%, 93%, 95%, 97% or 99% identical to the polypeptide according to SEQ ID NO. 2 and have the activity mentioned.

The invention furthermore relates to a process for the fermentative preparation of amino acids chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine using Coryneform bacteria which, in particular, already produce amino acids and in which the nucleotide sequences which code for the tmk gene are attenuated, in particular eliminated or expressed at a low level.

The microorganisms to which the present invention relates can prepare amino acids from glucose, sucrose, lactose, fructose, maltose, molasses, starch, cellulose or from glycerol and ethanol. They can be representatives of Coryneform bacteria, in particular of the genus Corynebacterium. Of the genus Corynebacterium, there may be mentioned in particular the species Corynebacterium glutamicum, which is known among experts for its ability to produce L-amino acids.

Suitable strains of the genus Corynebacterium, in particular of the species Corynebacterium glutamicum (C. glutamicum), are in particular the known wild-type strains

Corynebacterium glutamicum ATCC13032

Corynebacterium acetoglutamicum ATCC15806

Corynebacterium acetoacidophilum ATCC 13870

Corynebacterium melassecola ATCC17965

Corynebacterium thermoaminogenes FERM BP-1539

Brevibacterium flavum ATCC14067

Brevibacterium lactofermentum ATCC13869 and

Brevibacterium divaricatum ATCC14020 or L-amino acid-producing mutants or strains prepared therefrom.

Preferably, a bacterial strand with attenuated expression of tmk gene products with thymidylate kinase activity will improve amino acid yields at least 1%.

The inventors have isolated the new tmk gene from C. glutamicum, which codes for thymidylate kinase (EC 2.7.4.9).

To isolate the tmk gene or also other genes of C. glutamicum, a gene library of this microorganism is first set up in Escherichia coli (E. coli). The setting up of gene libraries is described in generally known textbooks and handbooks. The textbook by Winnacker: Gene und Klone, Eine Einfürung in die Gentechnologie [Genes and Clones, An Introduction to Genetic Engineering] (Verlag Chemie, Weinheim, Germany, 1990), or the handbook by Sambrook et al.: Molecular Cloning, A Laboratory Manual (Cold Spring Harbor Laboratory Press, 1989) may be mentioned as an example. A well-known gene library is that of the E. coli K-12 strain W3110 set up in λ vectors by Kohara et al. (Cell 50, 495-508 (1987)). Bathe et al. (Molecular and General Genetics, 252:255-265, 1996) describe a gene library of C. glutamicum ATCC13032, which was set up with the aid of the cosmid vector SuperCos I (Wahl et al., 1987, Proceedings of the National Academy of Sciences USA, 84:2160-2164) in the E. coli K-12 strain NM554 (Raleigh et al., 1988, Nucleic Acids Research 16:1563-1575). Börmann et al. (Molecular Microbiology 6(3), 317-326)) (1992)) in turn describe a gene library of C. glutamicum ATCC13032 using the cosmid pHC79 (Hohn and Collins, 1980, Gene 11, 291-298).

To prepare a gene library of C. glutamicum in E. coli, it is also possible to use plasmids such as pBR322 (Bolivar, 1979, Life Sciences, 25, 807-818) or pUC9 (Vieira et al., 1982, Gene, 19:259-268). Suitable hosts are, in particular, those E. coli strains which are restriction- and recombination-defective, such as the strain DH5αmcr, which has been described by Grant et al. (Proceedings of the National Academy of Sciences USA, 87 (1990) 4645-4649).

The long DNA fragments cloned with the aid of cosmids or other λ vectors can then in turn be subcloned and subsequently sequenced in the usual vectors which are suitable for DNA sequencing, such as is described e.g. by Sanger et al. (Proceedings of the National Academy of Sciences of the United States of America, 74:5463-5467, 1977).

The resulting DNA sequences can then be investigated with known algorithms or sequence analysis programs, such as e.g. that of Staden (Nucleic Acids Research 14, 217-232(1986)), that of Marck (Nucleic Acids Research 16, 1829-1836 (1988)) or the GCG program of Butler (Methods of Biochemical Analysis 39, 74-97 (1998)).

The new DNA sequence of C. glutamicum which codes for the tmk gene and which, as SEQ ID NO. 1, is a constituent of the present invention has been found in this manner. The amino acid sequence of the corresponding protein has furthermore been derived from the present DNA sequence by the methods described above. The resulting amino acid sequence of the tmk gene product is shown in SEQ ID NO. 2.

Coding DNA sequences, which result from SEQ ID NO. 1 by the degeneracy of the genetic code, are also a constituent of the invention. In the same way, DNA sequences, which hybridize with SEQ ID NO. 1 or parts of SEQ ID NO. 1, are a constituent of the invention. Conservative amino acid exchanges, such as e.g. exchange of glycine for alanine or of aspartic acid for glutamic acid in proteins, are furthermore known among experts as “sense mutations” which do not lead to a fundamental change in the activity of the protein, i.e. are of neutral function. It is furthermore known that changes on the N and/or C terminus of a protein cannot substantially impair or can even stabilize the function thereof. Information in this context can be found by the expert, inter alia, in Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in known textbooks of genetics and molecular biology. Amino acid sequences, which result in a corresponding manner from SEQ ID NO. 2, are also a constituent of the invention.

In the same way, DNA sequences, which hybridize with SEQ ID NO. 1 or parts of SEQ ID NO. 1, are a constituent of the invention. Finally, DNA sequences, which are prepared by the polymerase chain reaction (PCR) using primers, which result from SEQ ID NO. 1, are a constituent of the invention. Such oligonucleotides typically have a length of at least 15 nucleotides.

The skilled artisan will find instructions for identifying DNA sequences by means of hybridization can be found by the expert, inter alia, in the handbook “The DIG System Users Guide for Filter Hybridization” from Boehringer Mannheim GmbH (Mannheim, Germany, 1993) and in Liebl et al. (International Journal of Systematic Bacteriology 41: 255-260 (1991)). The hybridization takes place under stringent conditions, that is to say only hybrids in which the probe and target sequence, i.e. the polynucleotides treated with the probe, are at least 70% identical are formed. It is known that the stringency of the hybridization, including the washing steps, is influenced or determined by varying the buffer composition, the temperature and the salt concentration. The hybridization reaction is preferably carried out under a relatively low stringency compared with the washing steps (Hybaid Hybridisation Guide, Hybaid Limited, Teddington, UK, 1996).

A 5× SSC buffer at a temperature of approx. 50° C. -68° C., for example, can be employed for the hybridization reaction. Probes can also hybridize here with polynucleotides, which are less than 70% identical to the sequence of the probe. Such hybrids are less stable and are removed by washing under stringent conditions. This can be achieved, for example, by lowering the salt concentration to 2× SSC and optionally subsequently 0.5× SSC (The DIG System User's Guide for Filter Hybridisation, Boehringer Mannheim, Mannheim, Germany, 1995) a temperature of approx. 50° C. -68° C. being established. It is optionally possible to lower the salt concentration to 0.1× SSC.

Polynucleotide fragments which are, for example, at least 70% or at least 80% or at least 90% to 95% identical to the sequence of the probe employed can be isolated by increasing the hybridization temperature stepwise from 50° C. to 68° C. in steps of approx. 1-2° C. Further instructions on hybridization are obtainable on the market in the form of so-called kits (e.g. DIG Easy Hyb from Roche Diagnostics GmbH, Mannheim, Germany, Catalogue No. 1603558).

A skilled artisan will find instructions for amplification of DNA sequences with the aid of the polymerase chain reaction (PCR) can be found by the expert, inter alia, in the handbook by Gait: Oligonucleotide Synthesis: A Practical Approach (IRL Press, Oxford, UK, 1984) and in Newton and Graham: PCR (Spektrum Akademischer Verlag, Heidelberg, Germany, 1994).

The inventors have shown that Coryneform bacteria produce amino acids in an improved manner after attenuation of the tmk gene.

To achieve attenuation, either the expression of the tmk gene or the catalytic properties of the enzyme protein can be reduced or eliminated. The two measures can optionally be combined.

The reduction in gene expression can take place by suitable culturing or by genetic modification (mutation) of the signal structures of gene expression. Signal structures of gene expression are, for example, repressor genes, activator genes, operators, promoters, attenuators, ribosome binding sites, the start codon and terminators. The expert can find information on this e.g. in WO 96/15246, in Boyd and Murphy (Journal of Bacteriology 170: 5949 (1988)), in Voskuil and Chambliss (Nucleic Acids Research 26: 3548 (1998), in Jensen and Hammer (Biotechnology and Bioengineering 58: 191 (1998)), in Patek et al. (Microbiology 142: 1297 (1996)), Vasicova et al. (Journal of Bacteriology 181: 6188 (1999)) and in known textbooks of genetics and molecular biology, such as e.g. the textbook by Knippers (“Molekulare Genetik [Molecular Genetics]”, 6th edition, Georg Thieme Verlag, Stuttgart, Germany, 1995) or that by Winnacker (“Gene und Klone [Genes and Clones]”, VCH Verlagsgesellschaft, Weinheim, Germany, 1990).

Mutations which lead to a change or reduction in the catalytic properties of enzyme proteins are known from the prior art; examples which may be mentioned are the works by Qiu and Goodman (Journal of Biological Chemistry 272: 8611-8617 (1997)), Sugimoto et al. (Bioscience Biotechnology and Biochemistry 61: 1760-1762 (1997)) and Mockel (“Die Threonindehydratase aus Corynebacterium glutamicum: Aufhebung der allosterischen Regulation und Struktur des Enzyms [Threonine dehydratase from Corynebacterium glutamicum: Canceling the allosteric regulation and structure of the enzyme]”, Reports from the Jülich Research Center, Jül -2906, ISSN09442952, Jülich, Germany, 1994). Summarizing descriptions can be found in known textbooks of genetics and molecular biology, such as e.g. that by Hagemann (“Allgemeine Genetik [General Genetics]”, Gustav Fischer Verlag, Stuttgart, 1986).

Possible mutations are transitions, transversions, insertions and deletions. These mutations may be referred to as “missense mutations” or “nonsense mutations”, depending on the effect of the amino acid exchange on the enzyme activity. Insertions or deletions of at least one base pair (bp) in a gene lead to frame shift mutations, as a consequence of which incorrect amino acids are incorporated or translation is interrupted prematurely. Deletions of several codons typically lead to a complete loss of the enzyme activity. Instructions on generation of such mutations are prior art and can be found in known textbooks of genetics and molecular biology, such as e.g. the textbook by Knippers (“Molekulare Genetik [Molecular Genetics]”, 6th edition, Georg Thieme Verlag, Stuttgart, Germany, 1995), that by Winnacker (“Gene und Klone [Genes and Clones]”, VCH Verlagsgesellschaft, Weinheim, Germany, 1990) or that by Hagemann (“Allgemeine Genetik [General Genetics]”, Gustav Fischer Verlag, Stuttgart, 1986).

A common method of mutating genes of C. glutamicum is the method of “gene disruption” and “gene replacement” described by Schwarzer and Püthler (Bio/Technology 9, 84-87 (1991)).

In the method of gene disruption a central part of the coding region of the gene of interest is cloned in a plasmid vector which can replicate in a host (typically E. coli), but not in C. glutamicum. Possible vectors are, for example, pSUP301 (Simon et al., Bio/Technology 1, 784-791 (1983)), pK18mob or pK19mob (Schäfer et al., Gene 145, 69-73 (1994)), pK18mobsacB or pK19mobsacB (Jäger et al., Journal of Bacteriology 174: 5462-65 (1992)), pGEM-T (Promega Corporation, Madison, Wis., USA), pCR2.1-TOPO (Shuman (1994). Journal of Biological Chemistry 269:32678-84; U.S. Pat. No. 5,487,993), pCR®Blunt (Invitrogen, Groningen, Holland; Bernard et al., Journal of Molecular Biology, 234: 534-541 (1993)) or pEM1 (Schrumpf et al, 1991, Journal of Bacteriology 173:4510-4516). The plasmid vector, which contains the central part of the coding region of the gene, is then transferred into the desired strain of C. glutamicum by conjugation or transformation. The method of conjugation is described, for example, by Schafer et al. (Applied and Environmental Microbiology 60, 756-759 (1994)). Methods for transformation are described, for example, by Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a “cross-over” event, the coding region of the gene in question is interrupted by the vector sequence and two incomplete alleles are obtained, one lacking the 3′ end and one lacking the 5′ end. This method has been used, for example, by Fitzpatrick et al. (Applied Microbiology and Biotechnology 42, 575-580 (1994)) to eliminate the recA gene of C. glutamicum.

In the method of “gene replacement”, a mutation, such as e.g. a deletion, insertion or base exchange, is established in vitro in the gene of interest. The allele prepared is in turn cloned in a vector, which is not replicative for C. glutamicum, and this is then transferred into the desired host of C. glutamicum by transformation or conjugation. After homologous recombination by means of a first “cross-over” event which effects integration and a suitable second “cross-over” event which effects excision in the target gene or in the target sequence, the incorporation of the mutation or of the allele is achieved. This method was used, for example, by Peters-Wendisch et al. (Microbiology 144, 915-927 (1998)) to eliminate the pyc gene of C. glutamicum by a deletion.

A deletion, insertion or a base exchange can be incorporated into the tmk gene in this manner.

In addition, it may be advantageous for the production of L-amino acids to enhance, in particular over-express, one or more enzymes of the particular biosynthesis pathway, of glycolysis, of anaplerosis, of the citric acid cycle, of the pentose phosphate cycle, of amino acid export and optionally regulatory proteins, in addition to the attenuation of the tmk gene.

By enhancement measures, in particular over-expression, the activity or concentration of the corresponding protein is in general increased by at least 10%, 25%, 50%, 75%, 100%, 150%, 200%, 300%, 400% or 500%, up to a maximum of 1000% or 2000%, based on that of the wild-type protein or the activity or concentration of the protein in the starting microorganism.

Thus, for example, for the preparation of L-amino acids, in addition to the attenuation of the tmk gene at the same time one or more of the genes chosen from the group consisting of:

(a) the dapA gene which codes for dihydrodipicolinate synthase (EP-B 0 197 335),

(b) the gap gene which codes for glyceraldehyde 3-phosphate dehydrogenase (Eikmanns (1992), Journal of Bacteriology 174: 6076-6086),

(c) the tpi gene which codes for triose phosphate isomerase (Eikmanns (1992), Journal of Bacteriology 174:6076-6086),

(d) the pgk gene which codes for 3-phosphoglycerate kinase (Eikmanns (1992), Journal of Bacteriology 174:6076-6086),

(e) the zwf gene which codes for glucose 6-phosphate dehydrogenase (JP-A-09224661),

(f) the pyc gene which codes for pyruvate carboxylase (DE-A-198 31 609),

(g) the mqo gene which codes for malate-quinone oxidoreductase (Molenaar et al., European Journal of Biochemistry 254, 395-403 (1998)),

(h) the lysC gene which codes for a feed-back resistant aspartate kinase (Accession No.P26512; EP-B-0387527; EP-A-0699759),

(i) the lysE gene which codes for lysine export (DE-A-195 48 222),

(j) the hom gene which codes for homoserine dehydrogenase (EP-A 0131171),

(k) the ilvA gene which codes for threonine dehydratase (Mockel et al., Journal of Bacteriology (1992) 8065-8072)) or the ilvA(Fbr) allele which codes for a “feed back resistant” threonine dehydratase (Mockel et al., (1994) Molecular Microbiology 13: 833-842),

(l) the ilvBN gene which codes for acetohydroxy-acid synthase (EP-B 0356739),

(m) the ilvD gene which codes for dihydroxy-acid dehydratase (Sahm and Eggeling (1999) Applied and Environmental Microbiology 65: 1973-1979),

(n) the zwa1 gene which codes for the Zwal protein (DE: 19959328.0, DSM 13115), may be enhanced and, in particular, over-expressed.

Furthermore, it may be advantageous for the production of amino acids, in addition to the attenuation of the tmk gene, at the same time for one or more of the genes chosen from the group consisting of:

(a) the pck gene which codes for phosphoenol pyruvate carboxykinase (DE 199 50 409.1, DSM 13047),

(b) the pgi gene which codes for glucose 6-phosphate isomerase (US 09/396,478, DSM 12969),

(c) the poxB gene which codes for pyruvate oxidase (DE: 1995 1975.7, DSM 13114),

(d) the zwa2 gene which codes for the Zwa2 protein (DE: 19959327.2, DSM 13113), to be attenuated and, in particular, for the expression thereof to be reduced.

In addition to the attenuation of the tmk gene it may furthermore be advantageous for the production of amino acids to eliminate undesirable side reactions (Nakayama: “Breeding of Amino Acid Producing Microorganisms”, in: Overproduction of Microbial Products, Krumphanzl, Sikyta, Vanek (eds.), Academic Press, London, UK, 1982).

The invention also provides the microorganisms prepared according to the invention, and these can be cultured continuously or discontinuously in the batch process (batch culture) or in the fed batch (feed process) or repeated fed batch process (repetitive feed process) for the purpose of production of L-amino acids. A summary of known culture methods is described in the textbook by Chmiel (Bioprozesstechnik 1. Einführung in die Bioverfahrenstechnik [Bioprocess Technology 1. Introduction to Bioprocess Technology (Gustav Fischer Verlag, Stuttgart, 1991)) or in the textbook by Storhas (Bioreaktoren und periphere Einrichtungen [Bioreactors and Peripheral Equipment] (Vieweg Verlag, Braunschweig/Wiesbaden, 1994)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981).

The substances:

(a) sugars and carbohydrates, such as e.g. glucose, sucrose, lactose, fructose, maltose, molasses, starch and cellulose,

(b) oils and fats, such as, soya oil, sunflower oil, groundnut oil and coconut fat,

(c) fatty acids, such as palmitic acid, stearic acid and linoleic acid,

(d) alcohols, such as glycerol and ethanol, and

(e) organic acids, such as acetic acid, may be used individually, or as a mixture, as the source of carbon.

The substances:

(a) Organic nitrogen-containing compounds, such as peptones, yeast extract, meat extract, malt extract, corn steep liquor, soya bean flour and urea, or

(b) inorganic compounds, such as ammonium sulfate, ammonium chloride, ammonium phosphate, ammonium carbonate and ammonium nitrate, can be used be used individually, or as a mixture, as the source of nitrogen.

Phosphoric acid, potassium dihydrogen phosphate or dipotassium hydrogen phosphate or the corresponding sodium-containing salts can be used as the source of phosphorus.

The culture medium must furthermore comprise salts of metals, such as magnesium sulfate or iron sulfate, which are necessary for growth.

Essential growth substances, such as amino acids and vitamins, can be employed in addition to the above-mentioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH of the culture.

Antifoams, such as, for example, fatty acid polyglycol esters, can be employed to control the development of foam. Suitable substances having a selective action, such as, for example, antibiotics, can be added to the medium to maintain the stability of plasmids. To maintain aerobic conditions, oxygen or oxygen-containing gas mixtures, such as air, are introduced into the culture. The temperature of the culture is usually 20° C. to 45° C., and preferably 25° C. to 40° C. Culturing is continued until a maximum of the desired product has formed. This target is usually reached within 10 hours to 160 hours.

Methods for the determination of L-amino acids are known from the prior art. The analysis can thus be carried out, for example, as described by Spackman et al. (Analytical Chemistry, 30, (1958), 1190) by anion exchange chromatography with subsequent ninhydrin derivation, or it can be carried out by reversed phase HPLC, for example as described by Lindroth et al. (Analytical Chemistry (1979) 51: 1167-1174).

The process according to the invention is used for fermentative preparation of amino acids.

The isolation of plasmid DNA from Escherichia coli and all techniques of restriction, Klenow and alkaline phosphatase treatment were carried out by the method of Sambrook et al. (Molecular Cloning. A Laboratory Manual, 1989, Cold Spring Harbor Laboratory Press, Cold Spring Harbor, N.Y., USA). Methods for transformation of Escherichia coli are also described in this handbook.

The composition of the usual nutrient media, such as LB or TY medium, can also be found in the handbook by Sambrook et al.

The following microorganisms were deposited as pure cultures on Jul. 31, 2001 at the Deutsche Sammlung für Mikroorganismen und Zellkulturen (DSMZ=German Collection of Microorganisms and Cell Cultures, Braunschweig, Germany) in accordance with the Budapest Treaty:

Escherichia coli DH5alphamcr/pXK99E (=DH5αmcr/pXK99E) as DSM 14440,

Escherichia coli DH5alphamcr/pXK99Etmk (=DH5αmcr/pXK99Etmk) as DSM 14439.

The present invention is explained in more detail with the aid of the following embodiment examples.

### EXAMPLES

The abbreviations and designations have the following meaning.

| | |
|---|---|
| Kan: | Kanamycin resistance gene aph(3′)-IIa from Escherichia coli |
| KpnI | Cleavage site of the restriction enzyme KpnI |
| NcoI | Cleavage site of the restriction enzyme NcoI |
| XbaI | Cleavage site of the restriction enzyme XbaI |
| Ptrc | trc promoter |
| T1 | Termination region T1 |
| T2 | Termination region T2 |
| LacIq | lacIq repressor of the lac operon of Escherichia coli |
| OriV | Replication origin ColE1 from E. coli |
| Tmk | Cloned region of the tmk gene |

### Example 1

Preparation of a genomic cosmid gene library from C. glutamicum ATCC 13032

Chromosomal DNA from C. glutamicum ATCC 13032 was isolated as described by Tauch et al. (1995, Plasmid 33:168-179) and partly cleaved with the restriction enzyme Sau3AI (Amersham Pharmacia, Freiburg, Germany, Product Description Sau3AI, Code no. 27-0913-02). The DNA fragments were dephosphorylated with shrimp alkaline phosphatase (Roche Molecular Biochemicals, Mannheim, Germany, Product Description SAP, Code no. 1758250). The DNA of the cosmid vector SuperCos1 (Wahl et al. (1987), Proceedings of the National Academy of Sciences, USA 84:2160-2164), obtained from Stratagene (La Jolla, USA, Product Description SuperCos1 Cosmid Vector Kit, Code no. 251301) was cleaved with the restriction enzyme XbaI (Amersham Pharmacia, Freiburg, Germany, Product Description XbaI, Code no. 27-0948-02) and likewise dephosphorylated with shrimp alkaline phosphatase.

The cosmid DNA was then cleaved with the restriction enzyme BamHI (Amersham Pharmacia, Freiburg, Germany, Product Description BamHI, Code no. 27-0868-04). The cosmid DNA treated in this manner was mixed with the treated ATCC13032 DNA and the batch was treated with T4 DNA ligase (Amersham Pharmacia, Freiburg, Germany, Product Description T4-DNA-Ligase, Code no.27-0870-04). The ligation mixture was then packed in phages with the aid of Gigapack II XL Packing Extracts (Stratagene, La Jolla, USA, Product Description Gigapack II XL Packing Extract, Code no. 200217).

For infection of the E. coli strain NM554 (Raleigh et al. 1988, Nucleic Acid Res. 16:1563-1575) the cells were taken up in 10 mM MgSO₄ and mixed with an aliquot of the phage suspension. The infection and titering of the cosmid library were carried out as described by Sambrook et al. (1989, Molecular Cloning: A Laboratory Manual, Cold Spring Harbor), the cells being plated out on LB agar (Lennox, 1955, Virology, 1:190)+100 μg/ml ampicillin. After incubation overnight at 37° C., recombinant individual clones were selected.

### Example 2

Isolation and Sequencing of the tmk Gene

The cosmid DNA of an individual colony was isolated with the Qiaprep Spin Miniprep Kit (Product No. 27106, Qiagen, Hilden, Germany) in accordance with the manufacturer's instructions and partly cleaved with the restriction enzyme Sau3AI (Amersham Pharmacia, Freiburg, Germany, Product Description Sau3AI, Product No. 27-0913-02). The DNA fragments were dephosphorylated with shrimp alkaline phosphatase (Roche Molecular Biochemicals, Mannheim, Germany, Product Description SAP, Product No. 1758250). After separation by gel electrophoresis, the cosmid fragments in the size range of 1500 to 2000 bp were isolated with the QiaExII Gel Extraction Kit (Product No. 20021, Qiagen, Hilden, Germany).

The DNA of the sequencing vector pZero-1, obtained from Invitrogen (Groningen, Holland, Product Description Zero Background Cloning Kit, Product No. K2500-01) was cleaved with the restriction enzyme BamHI (Amersham Pharmacia, Freiburg, Germany, Product Description BamHI, Product No. 27-0868-04). The ligation of the cosmid fragments in the sequencing vector pZero-1 was carried out as described by Sambrook et al. (1989, Molecular Cloning: A Laboratory Manual, Cold Spring Harbor), the DNA mixture being incubated overnight with T4 ligase (Pharmacia Biotech, Freiburg, Germany). This ligation mixture was then electroporated (Tauch et al. 1994, FEMS Microbiol. Letters, 123:343-7) into the E. coli strain DH5αmcr (Grant, 1990, Proceedings of the National Academy of Sciences, U.S.A., 87:4645-4649). Letters, 123:343-7) and plated out on LB agar (Lennox, 1955, Virology, 1:190) with 50 mg/l zeocin.

The plasmid preparation of the recombinant clones was carried out with the Biorobot 9600 (Product No. 900200, Qiagen, Hilden, Germany). The sequencing was carried out by the dideoxy chain termination method of Sanger et al. (1977, Proceedings of the National Academy of Sciences, U.S.A., 74:5463-5467) with modifications according to Zimmermann et al. (1990, Nucleic Acids Research, 18:1067). The “RR dRhodamin Terminator Cycle Sequencing Kit” from PE Applied Biosystems (Product No. 403044, Weiterstadt, Germany) was used. The separation by gel electrophoresis and analysis of the sequencing reaction were carried out in a “Rotiphoresis NF Acrylamide/Bisacrylamide” Gel (29: 1) (Product No. A124.1, Roth, Karlsruhe, Germany) with the “ABI Prism 377” sequencer from PE Applied Biosystems (Weiterstadt, Germany).

The raw sequence data obtained were then processed using the Staden program package (1986, Nucleic Acids Research, 14:217-231) version 97-0. The individual sequences of the pZero1 derivatives were assembled to a continuous contig. The computer-assisted coding region analyses were prepared with the XNIP program (Staden, 1986, Nucleic Acids Research, 14:217-231). Further analyses were carried out with the “BLAST search programs” (Altschul et al., 1997, Nucleic Acids Research, 25:3389-3402) against the non-redundant databank of the “National Center for Biotechnology Information” (NCBI, Bethesda, Md., USA).

The resulting nucleotide sequence is shown in SEQ ID NO. 1. Analysis of the nucleotide sequence showed an open reading frame of 612 bp, which was called the tmk gene. The tmk gene codes for a polypeptide of 203 amino acids (See SEQ ID NO. 2)

### Example 3

Preparation of the expression vector pXK99Etmk for IPTG-induced expression of the tmk gene in C. glutamicum

3.1 Cloning of the tmk Gene

From the strain ATCC 13032, chromosomal DNA was isolated by the method of Eikmanns et al. (Microbiology 140: 1817-1828 (1994)). On the basis of the sequence of the tmk gene known for C. glutamicum from example 2, the following oligonucleotides were chosen for the polymerase chain reaction:

| | |
|---|---|
| (i) | 5′-TG GGT ACC-ATT CAA GCC GGA GCA CTA CC-3′ (SEQ ID NO. 3), |
| | and |
| (ii) | 5′-GA TCT AGA-CAG CGC CGA ATC CGA TTC AT-3′ (SEQ ID NO. 4) |

The primers were chosen here so that the amplified fragment contains the incomplete gene, starting with the native ribosome binding site without the promoter region, and the front region of the tmk gene. Furthermore, the primer sequence of SEQ ID NO. 3 contains the sequence for the cleavage site of the restriction endonuclease Kpn 1. The primer SEQ ID NO. 4 contains the sequence for the cleavage site of the restriction endonuclease XbaI. The respective restriction endonuclease cleavage sites in both SEQ ID NO. 3 and SEQ ID NO. 4 are marked by underlining in the nucleotide sequences shown above.

The primers shown were synthesized by MWG-Biotech AG (Ebersberg, Germany) and the PCR reaction was carried out by the standard PCR method of Innis et al. (PCR Protocols. A Guide to Methods and Applications, 1990, Academic Press) with Pwo-Polymerase from Roche Diagnostics GmbH (Mannheim, Germany). With the aid of the polymerase chain reaction, the primers allow amplification of a DNA fragment 520 bp in size, which carries the incomplete tmk gene, including the native ribosome binding site. The tmk fragment 520 bp in size was cleaved with the restriction endonucleases KpnI and XbaI and then isolated from the agarose gel with the QiaExII Gel Extraction Kit (Product No. 20021, Qiagen, Hilden, Germany).

3.2 Construction of the Expression Vector pXK99E

The IPTG-inducible expression vector pXK99E was constructed according to the prior art. The vector is based on the Escherichia coli expression vector pTRC99A (Amann et al., Gene 69: 301-315 (1988)) and contains the trc promoter, which can be induced by addition of the lactose derivative IPTG (isopropyl β-D-thiogalactopyranoside), the termination regions T1 and T2, the replication origin ColE1 from E. coli, the lacI^{q} gene (repressor of the lac operon from E. coli), a multiple cloning site (mcs) (Norrander, J. M. et al. Gene 26, 101-106 (1983)) and the kanamycin resistance gene aph(3 ′)-IIa from E. coli (Beck et al. (1982), Gene 19: 327-336).

It has been found the vector pXK99E is quite specifically suitable for regulating the expression of a gene, in particular effecting attenuated expression in Coryneform bacteria. The vector pXK99E is an E. coli expression vector and can be employed in E. coli for enhanced expression of a gene.

Since the vector cannot replicate independently in Coryneform bacteria, this is retained in the cell only if it is integrated into the chromosome. The peculiarity of this vector here is the use for regulated expression of a gene after cloning of a gene section from the front region of the corresponding gene in the vector containing the start codon and the native ribosome binding site, and subsequent integration of the vector into Coryneform bacteria, in particular C. glutamicum. Gene expression is regulated by addition of metered amounts of IPTG to the nutrient medium. Amounts of 1 μM/l up to 10 μM/l IPTG have the effect of very weak expression of the corresponding gene, and amounts of 10 μM/l up to 100 μM/l have the effect of a slightly attenuated to normal expression of the corresponding gene.

The E. coli expression vector pXK99E constructed was transferred by means of electroporation (Tauch et al. 1994, FEMS Microbiol Letters, 123: 343-347) into E. coli DH5αmcr (Grant, 1990, Proceedings of the National Academy of Sciences U.S.A., 87:4645-4649). Selection of the transformants was carried out on LB Agar (Sambrook et al., Molecular Cloning: A Laboratory Manual. 2^{nd} Ed., Cold Spring Harbor Laboratory Press, Cold Spring Harbor, N.Y., 1989), which had been supplemented with 50 mg/l kanamycin. Plasmid DNA was isolated from a transformant by conventional methods (Peters-Wendisch et al., 1998, Microbiology, 144, 915-927), cleaved with the restriction endonuclease NcoI, and the plasmid was checked by subsequent agarose gel electrophoresis.

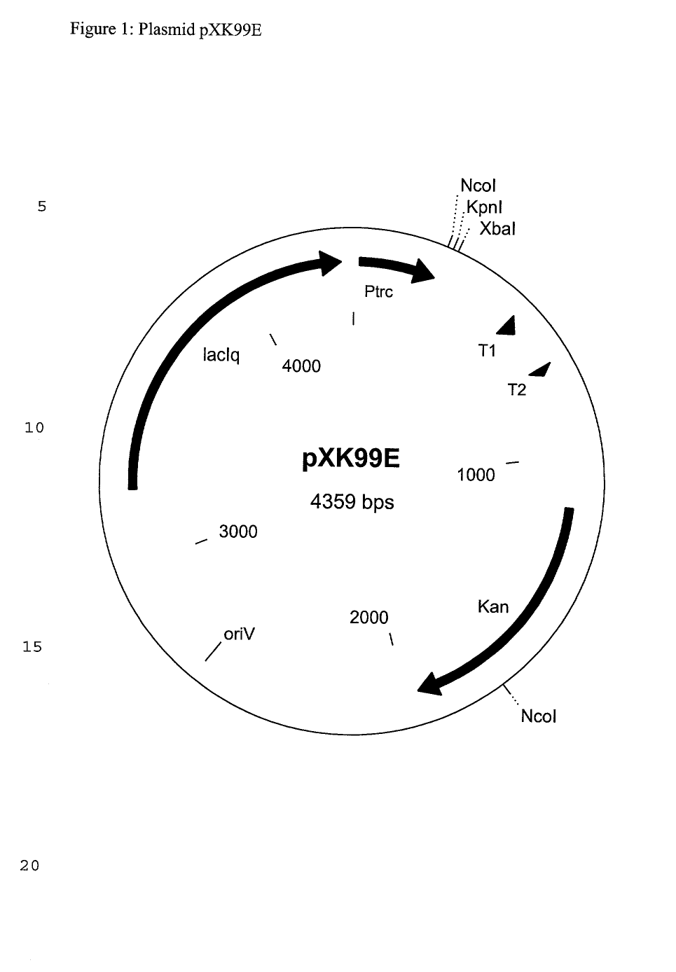

The plasmid construct obtained in this way was called pXK99E (FIG. 1). The strain obtained by electroporation of the plasmid pXK99E in the E. coli strain DH5αmcr was called E. coli DH5alphamcr/pXK99E (=DH5αmcr/pXK99E) and deposited on Jul. 31, 2001 as DSM 14440 at the Deutsche Sammlung für Mikroorganismen und Zellkulturen (DSMZ=German Collection of Microorganisms and Cell Cultures, Braunschweig, Germany) in accordance with the Budapest Treaty.

3.3 Cloning of the tmk Fragment in the E. coli Expression Vector pXK99E

The E. coli expression vector pXK99E described in example 3.2 was used as the vector. DNA of this plasmid was cleaved completely with the restriction enzymes KpnI and XbaI and then dephosphorylated with shrimp alkaline phosphatase (Roche Diagnostics GmbH, Mannheim, Germany, Product Description SAP, Product No. 1758250).

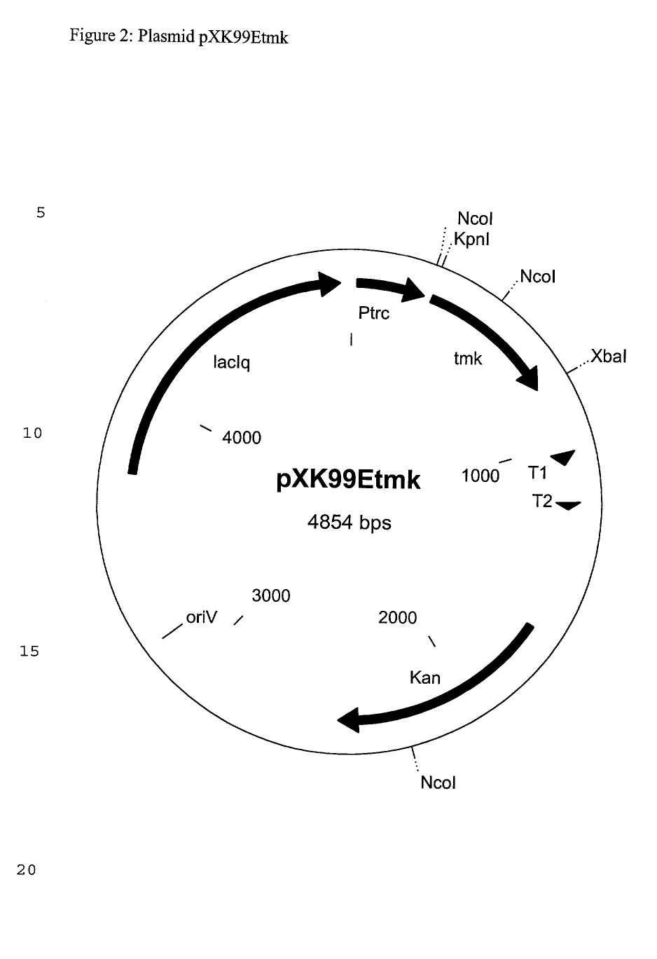

The tmk fragment, approximately 500 bp in size, described in example 3.1, obtained by means of PCR, and cleaved with the restriction endonucleases KpnI and XbaI was mixed with the prepared vector pXK99E. The batch was treated with T4 DNA ligase (Amersham Pharmacia, Freiburg, Germany, Product Description T4-DNA-Ligase, Code no.27-0870-04). The ligation batch was transformed in the E. coli strain DH5αmcr (Hanahan, In: DNA cloning. A Practical Approach. Vol. 1, IRL-Press, Oxford, Washington DC, USA). Selection of plasmid-carrying cells was made by plating out the transformation batch on LB agar (Lennox, 1955, Virology, 1:190) with 50 mg/l kanamycin. After incubation overnight at 37° C., recombinant individual clones were selected. Plasmid DNA was isolated from a transformant with the Qiaprep Spin Miniprep Kit (Product No. 27106, Qiagen, Hilden, Germany) in accordance with the manufacturer's instructions and cleaved with the restriction enzymes, KpnI and XbaI, to check the plasmid by subsequent agarose gel electrophoresis. The resulting plasmid was called pXK99Etmk. It is shown in FIG. 2.

### Example 4

Integration of the Vector pXK99Etmk into the Genome of the C. glutamicum Strain DSM5715

The vector pXK99E mentioned in Example 3 was electroporated by the electroporation method of Tauch et al.,(1989 FEMS Microbiology Letters 123: 343-347) in the strain C. glutamicum DSM5715. The vector cannot replicate independently in DSM5715 and is retained in the cell only if it has integrated into the chromosome. Selection of clones with integrated pXK99Etmk was carried out by plating out the electroporation batch on LB agar (Sambrook et al., Molecular Cloning: A Laboratory Manual. 2^{nd} Ed., Cold Spring Harbor, N.Y., 1989), which had been supplemented with 15 mg/l kanamycin and IPTG (1 mM/l).

For detection of the integration, the tmk fragment was labeled with the Dig hybridization kit from Boehringer by the method of “The DIG System Users Guide for Filter Hybridization” of Boehringer Mannheim GmbH (Mannheim, Germany, 1993). Chromosomal DNA of a potential integrant was isolated by the method of Eikmanns et al. (Microbiology 140: 1817-1828 (1994)) and in each case cleaved with the restriction enzymes NcoI and KpnI. The fragments formed were separated by means of agarose gel electrophoresis and hybridized at 68° C. with the Dig hybridization kit from Boehringer. The plasmid pXK99Etmk mentioned in example 3 had been inserted into the chromosome of DSM5715 within the chromosomal tmk gene. The strain was called DSM5715::pXK99Etmk.

### Example 5

Preparation of Lysine

The C. glutamicum strain DSM5715::pXK99Etmk obtained in example 4 was cultured in a nutrient medium suitable for the production of lysine and the lysine content in the culture supernatant was determined. By addition of IPTG (10 μM/l), attenuated expression of the tmk gene occurs, regulated by the trc promoter.

The strain was first incubated on an agar plate with the corresponding antibiotic (brain-heart agar with kanamycin (25 mg/l) and IPTG (10 μM/l)) for 24 hours at 33° C. Starting from this agar plate culture, a preculture was seeded (10 ml medium in a 100 ml conical flask). The complete medium CgIII was used as the medium for the preculture.

| | Cg III Medium |
|---|---|
| NaCl | 2.5 g/l |
| Bacto-Peptone | 10 g/l |
| Bacto-Yeast extract | 10 g/l |
| Glucose (autoclaved separately) | 2% (w/v) |
| The pH was adjusted to pH 7.4 | |

Kanamycin (25 mg/l) and IPTG (10 μM/l) were added to this. The preculture was incubated for 16 hours at 33° C. at 240 rpm on a shaking machine. A main culture was seeded from this preculture such that the initial OD (660 nm) of the main culture was 0.1 OD. Medium MM was used for the main culture.

MM Medium

| | | |
|---|---|---|
| CSL (corn steep liquor) | 5 | g/l |
| MOPS (morpholinopropanesulfonic acid) | 20 | g/l |
| Glucose (autoclaved separately) | 50 | g/l |
| Salts: | | |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * H₂O | 10 | mg/l |
| MnSO₄ * H₂O | 5.0 | mg/l |
| Biotin (sterile-filtered) | 0.3 | mg/l |
| Thiamine * HC1 (sterile-filtered) | 0.2 | mg/l |
| Leucine (sterile-filtered) | 0.1 | g/l |
| CaCO₃ | 25 | g/l |

The CSL, MOPS and the salt solution are brought to pH 7 with aqueous ammonia and autoclaved. The sterile substrate and vitamin solutions are then added, and the CaCO₃ autoclaved in the dry state is added.

Culturing is carried out in a 10 ml volume in a 100 ml conical flask with baffles. Kanamycin (25 mg/l) and IPTG (10 μM/l) were added. Culturing was carried out at 33° C. and 80% atmospheric humidity.

After 72 hours, the OD was determined at a measurement wavelength of 660 nm with a Biomek 1000 (Beckmann Instruments GmbH, Munich). The amount of lysine formed was determined with an amino acid analyzer from Eppendorf-BioTronik (Hamburg, Germany) by ion exchange chromatography and post-column derivation with ninhydrin detection.

The result of the experiment is shown in Table 1.

TABLE 1

| | OD | Lysine HCl |
|---|---|---|
| Strain | (660 nm) | g/l |
| DSM5715 | 7.8 | 13.58 |
| DSM5715::pXK99Etmk | 11.3 | 15.51 |

Numerous modifications and variations on the present invention are possible in light of the above teachings. It is, therefore, to be understood that within the scope of the accompanying claims, the invention may be practiced otherwise than as specifically described herein.

What is claimed is:

1. An isolated polynucleotide sequence, which encodes a polypeptide having the amino acid sequence of SEQ ID NO. 2.

2. The isolated polynucleotide sequence of claim 1, wherein said polypeptide sequence has thymidylate kinase activity.

3. A vector comprising the isolated polynucleotide sequence of claim 1.

4. A host cell comprising the isolated polynucleotide sequence of claim 1.

5. The host cell of claim 4, which is a Coryneform bacterium.

6. The host cell of claim 4, which is a Coryneform bacterium selected from the group consisting of Corynebacterium glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

7. A method for detecting polynucleotides with at least 70% homology to the polynucleotide of claim 1, comprising contacting a polynucleotide sample with a polynucleotide comprising at least 15 consecutive nucleotides of the polynucleotide sequence of claim 1, or at least 15 consecutive nucleotides of the complement thereof.

8. A method for producing polynucleotides with at least 70% homology to the polynucleotide of claim 1, comprising contacting a polynucleotide sample with a polynucleotide comprising at least 15 consecutive nucleotides of the polynucleotide sequence of claim 1, or at least 15 consecutive nucleotides of the complement thereof.

9. A process for screening for polynucleotide sequences, which encode a polypeptide having thymidylate kinase activity comprising
(a) hybridizing the isolated polynucleotide to claim 1 to a polynucleotide sample to be screened;
(b) expressing the polynucleotide to produce a polypeptide;
(c) detecting the presence or absence of thymidylate kinase activity of the polypeptide.

10. A method for making thymidylate kinase polypeptide, comprising
(a) culturing the host cell of claim 4 for a duration of time under conditions suitable for expression of thymidylate kinase polypeptide; and
(b) collecting the thymidylate kinase polypeptide.

11. An isolated polynucleotide, which comprises SEQ ID NO. 1.

12. An isolated polynucleotide, which is complementary to the polynucleotide of claim 11.

13. An isolated polynucleotide, which is at least 70% identical to the polynucleotide of claim 11.

14. An isolated polynucleotide, which is at least 80% identical to the polynucleotide of claim 11.

15. An isolated polynucleotide, which is at least 90% identical to the polynucleotide of claim 11.

16. An isolated polynucleotide, which comprises at least 15 consecutive nucleotides of the polynucleotide of claim 11.

17. An isolated polynucleotide, which hybridizes to the polynucleotide of claim 11.

18. The isolated polynucleotide of claim 11, which encodes a polypeptide having thymidylate kinase activity.

19. A vector comprising the isolated polynucleotide of claim 11.

20. A host cell comprising the isolated polynucleotide of claim 11.

21. The host cell of claim 20, which is a Coryneform bacterium.

22. The host cell of claim 20, which is a Coryneform bacterium selected from the group consisting of Corynebacterium glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

23. A process for screening for polynucleotide sequences, which encode a polypeptide having thymidylate kinase activity comprising
(a) hybridizing the isolated polynucleotide to claim 11 to a polynucleotide sample to be screened;
(b) expressing the polynucleotide to produce a polypeptide;
(c) detecting the presence or absence of thymidylate kinase activity of the polypeptide.

24. A method for detecting polynucleotides with at least 70% homology to the polynucleotide of claim 11, comprising contacting a polynucleotide sample with a polynucleotide comprising at least 15 consecutive nucleotides of the polynucleotide sequence of claim 11, or at least 15 consecutive nucleotides of the complement thereof.

25. A method for producing polynucleotides with at least 70% homology to the polynucleotide of claim 11, comprising contacting a polynucleotide sample with a polynucleotide comprising at least 15 consecutive nucleotides of the polynucleotide sequence of claim 11, or at least 15 consecutive nucleotides of the complement thereof.

26. A method for making thymidylate kinase polypeptide, comprising
(a) culturing the host cell of claim 20 for a duration of time under conditions suitable for expression of thymidylate kinase polypeptide; and
(b) collecting the thymidylate kinase polypeptide.

27. A Coryneform bacterium, which comprises attenuated expression of the tmk gene.

28. A Coryneform bacterium of claim 27, wherein the tmk gene comprises the polynucleotide sequence of SEQ ID NO. 1.

29. Escherichia coli DSM 14440.

30. Escherichia coli DSM 14439.

31. A process for producing L-amino acids comprising culturing a bacterial cell in a medium suitable for producing L-amino acids, wherein the bacterial cell comprises attenuated expression of the tmk gene.

32. The process of claim 31, wherein said bacterial cell is a Coryneform bacterium.

33. The process of claim 32, wherein the bacterial cell is a Coryneform bacterium from the group consisting of Corynebacterium glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

34. The process of claim 31, wherein the tmk gene comprises the polynucleotide sequence of SEQ ID NO. 1.

35. The process of claim 31, wherein the L-amino acid is L-lysine.

36. The process of claim 31, wherein the bacteria further comprises at least one gene whose expression is enhanced, wherein the gene is selected from the group consisting of dapA, gap, tpi, pgk, zwf, pyc, mqo, lysC, lysE, hom, ilvA, IlvA(Fbr), ilvBN, ilvD, and zwa1.

37. The process of claim 31, wherein the bacteria further comprises at least one gene whose expression is attenuated, wherein the gene is selected from the group consisting of pck, pgi, poxB, and zwa2.

38. An isolated polypeptide comprising the sequence of SEQ ID NO. 2.

39. An isolated polypeptide comprising an amino acid sequence, which is at least 70% identical to the peptide of claim 38.
